(19) United States
(12) Patent Application Publication
(10) Pub. No.: US 2017/0251979 A1
(43) Pub. Date: Sep. 7, 2017

(54) Camera in a Vehicle

(71) Applicant: Bayerische Motoren Werke Aktiengesellschaft, Muenchen (DE)

(72) Inventors: Matthias FRANZ, Brannenburg (DE); Victor ORLINSKIY, Muenchen (DE); Johannes RIEDEL, Muenchen (DE); Claudia SEDLMEIER, Landshut (DE)

(21) Appl. No.: 15/599,761

(22) Filed: May 19, 2017

Related U.S. Application Data
(63) Continuation of application No. PCT/EP2015/077043, filed on Nov. 19, 2015.

(30) Foreign Application Priority Data
Nov. 19, 2014 (DE) 10 2014 223 629.0

(51) Int. Cl. A61B 5/00; B60R 1/00; A61B 5/11; B60N 2/02; B60N 3/06; G06K 9/00; B60N 2/00

(52) U.S. Cl. CPC A61B 5/6893; G06K 9/00335; G06K 9/00845; B60R 1/00; B60N 2/002; B60N 2/0232; B60N 2/0244; B60N 3/06; A61B 5/0077; A61B 5/1128; A61B 5/7282; A61B 5/7405; A61B 5/742; B60R 2300/30; B60R 2300/8006; B60K 2350/2013; B60K 2350/106; B60K 2350/1096; B60K 2350/352; B60N 2002/0268; A61B 2576/00

(57) ABSTRACT

An arrangement is provided having a camera, wherein the camera is arranged in a vehicle. A movement of a user of the vehicle is determined by use of the camera and a processing device, wherein an interactive program is executable dependent on the movement of the user. Such an arrangement can provide signals which, for example, enable an interaction between the user and a system in the vehicle.

### CROSS REFERENCE TO RELATED APPLICATIONS

This application is a continuation of PCT International Application No. PCT/EP2015/077043, filed Nov. 19, 2015, which claims priority under 35 U.S.C. §119 from German Patent Application No. 10 2014 223 629.0, filed Nov. 19, 2014, the entire disclosures of which are herein expressly incorporated by reference.

### BACKGROUND AND SUMMARY OF THE INVENTION

The invention relates to a camera in a vehicle, particularly an arrangement having a camera.

It is known to provide a training function in a vehicle and to carry out an analysis of training movements by use of a pressure sensor system arranged in a seat.

It is an object of the invention to provide an arrangement for a vehicle, which creates an additional possibility of capturing inputs, for example, movements of a user in a vehicle. In particular, a control of components and procedural courses is to be made possible in this manner.

This and other objects are achieved according to the invention by an arrangement having a camera, wherein the camera is arranged in a vehicle and by way of the camera and a processing device, a user's movement can be determined in the vehicle. An interactive program can be implemented as a function of the user's movement.

The user may be a person who is sitting in a vehicle seat.

The processing unit is, for example, a control unit of the vehicle, which can carry out or activate various functions in the vehicle.

The vehicle may, for example, be one of the following vehicles: a motor vehicle, a passenger car, a truck, a bus or a motorcycle.

The processing unit supplies signals by way of image data of the at least one camera and permits an interaction between the user and a system of the vehicle which processes the signals. An input device is therefore provided, by which a user can generate signals for the interaction or for the control of the system.

The camera is, for example, a unit for capturing individual images or moving images. These can be captured in a digital or an analog form. The camera can, for example, photograph or film a user or a part of at least one user in the vehicle.

As a result, advantageously, a movement, for example, a change of the position of the user or of a part of the user can be captured. It is further contemplated to capture the user himself, for example, recognize who the user is, or in order to detect whether the user is already known, (for example, a user stored in a data bank).

The program is, for example, a training program in which the user carries out body training and the program is controlled as a function of image data of the camera and possibly additionally, further sensors, such as pressure sensors.

It is a further development that the camera is arranged in the vehicle such that the user is visually captured, and wherein image data of the camera can be analyzed by the processing unit of the vehicle.

The camera can, for example, be directed at a person sitting in a seat of the vehicle, for example, at the person's head or upper body.

A further development consists of the fact that image data of the camera and, additionally, signals of at least one pressure sensor can be analyzed.

The camera can therefore be used for capturing the user also by way of a pressure sensor system and, for example, determining his motions, his position or change of position. This permits the implementation of interactive programs, such as training programs.

It is a further development of the above that a condition of the user can be determined by use of the camera and the processing unit.

The user's condition may, for example, comprise or, for example, be based on

(i) at least one vital parameter of the user, such as body temperature, heart beat, body temperature, breathing, which can be determined by use of the image data of the camera;

(ii) the user's sitting position in a seat of the vehicle.

It is also a further development that the movement can be compared with at least one predefined value. For example, at least one desired value or threshold value can be specified, and various actions can be carried when that value is reached, exceeded or there is a falling below that value. The user's condition may also be compared with a predefined value and, as a function of such a comparison, an action can be carried out. The control of a training program, for example, can thereby be carried out.

The movement may particularly be a sequence of one or more position changes by the user or may, for example, include position changes of an extremity of the user in the course of time. The movement can therefore also be a sequence of movements, which can be evaluated, for example, by a training program.

For example, the user's condition and/or the movement can be matched with a user profile.

It is also a further development that at least one display unit and/or at least one loudspeaker can be activated by the processing unit.

For example, the user can carry out an interactive body training as a function of instructions, wherein the instructions can be displayed and/or emitted by way of the display unit and/or the loudspeaker.

It is a further development that an adjusting device can be activated as a function of the user's movement.

The adjusting device may be at least one actuator, which can be activated, for example by the processing device. The adjusting device may, for example, be a motor for adjusting a component of a seat.

It is a further development of the above, that the adjusting device is further developed for the user's training.

For example, a footrest may have an adjusting device which can be activated, which, for example, has a motor that can be controlled, and which builds up a counter-pressure onto a foot pressing against the footrest. Such an adjusting device is used, for example, for a training of the leg muscles. For example, interactive games or other programs can be implemented, in which a footrest is used as the input medium. Optionally, an actuator in the footrest can be activated by the processing unit such that the user receives feedback. Examples of an actuator activation may be a deflection, a vibration or a change of the force required for the interaction of the sensor unit (“force feedback”).

It is also a further development of the above that the adjusting device adjusts a seat position.

Thus, a seat position can be adjusted for a user who is captured by the camera while sitting in the seat.

It is a further development that a user can be identified by way of the camera, and a profile is downloaded that is assigned to this user. This makes it possible to implement programs individually for the user.

The above-mentioned object is also achieved by a motor vehicle having such an arrangement.

According to a further development, at least one program stored in a memory is assigned to the processing unit. Such a program can be controlled by means of the image data of the camera or signals of the other sensors. A program can also be used which, for example, for the implementation of a game or a communication, permits the interaction with at least one additional person inside and/or outside the vehicle. Such a communication may also be built up with other traffic participants.

Other objects, advantages and novel features of the present invention will become apparent from the following detailed description of one or more preferred embodiments when considered in conjunction with the accompanying drawings.

### BRIEF DESCRIPTION OF THE DRAWINGS

FIG. 1 is a view of a seat and of a control unit interacting with a camera in a vehicle.

FIG. 2 is a flow chart for a process which uses image data of the camera.

### DETAILED DESCRIPTION OF THE DRAWINGS

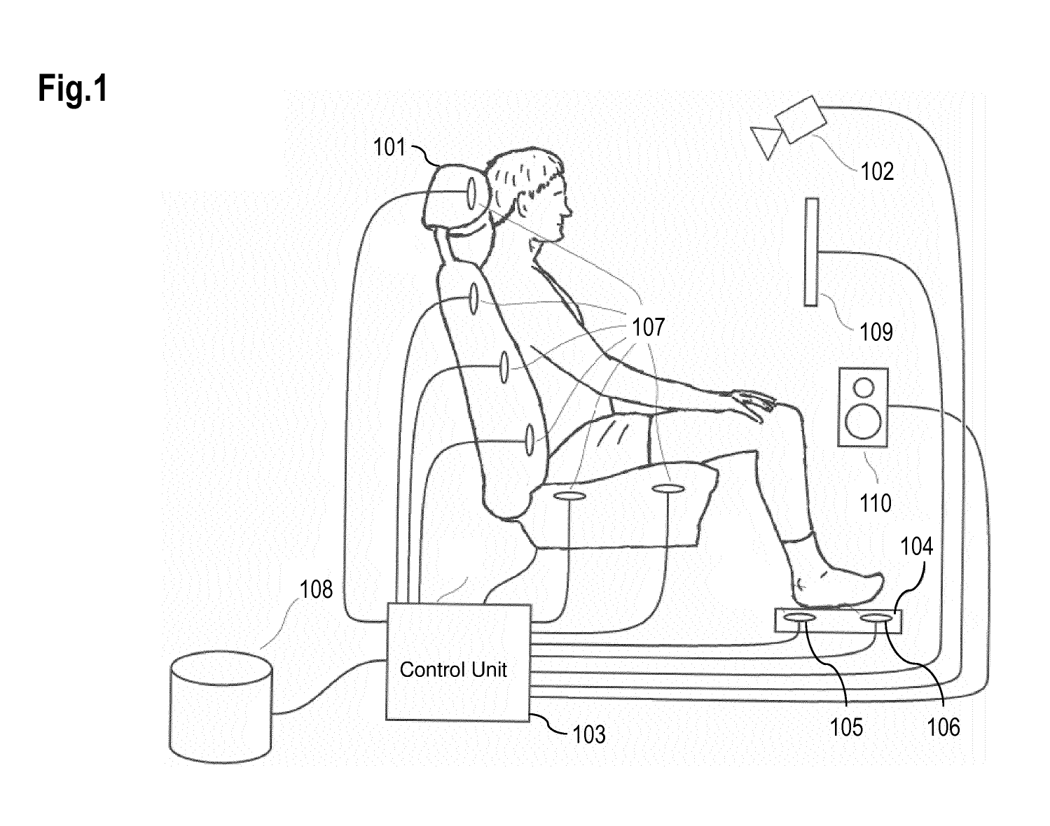

FIG. 1 illustrates a seat 101, which is mounted in a vehicle. A camera 102 is arranged in front of the seat. The camera 102 is connected to a control unit 103 of the vehicle. The camera 102 is directed at a person sitting in the seat 101, for example, at the person's head or upper body, and supplies corresponding image data to the control unit 103.

An optional footrest 104 with sensor units 105, 106 is arranged in front of the seat. By means of at least one foot, the person applies pressure to at least one sensor unit 105, 106. The signals of the sensor units 105, 106 are supplied to the control unit 103 of the vehicle.

The seat 101 has a plurality of optional pressure sensors 107, which detect a pressure that is applied to the pressure sensors 107 by the person sitting in the seat 101. Signals of the pressure sensors 107 are supplied to the control unit 103.

The signals of the sensor units 105, 106 and of the pressure sensors 107 can provide information concerning the type of actuation (such as duration, intensity, frequency, concerned surface, etc.).

An optional display unit 109 is connected to the control unit 103, which display unit 109 can be viewed by the person. Thus, as a function of the image data and of the signals of the sensor units 105, 106 and of the pressure sensors 107, a component on the display unit 109 can be influenced, for example, controlled, in order to supply to the person, as the user, with a visual acknowledgment concerning his sitting position, movements and his interaction with the sensor units 105, 106 and the pressure sensors 107.

A loudspeaker 110 may also be connected to the control unit 103, by way of which loudspeaker 110, for example, audio information can be emitted that was generated by the control unit 103.

In addition, a memory 108 (for example, in the form of a database or databank) is connected to the control unit 103, in which memory 108 programs can be stored which, for example, support an interaction with the sensor units 105 and the display unit 109.

The control unit 103 processes the received image data and the received signals and, as required, activates further components of the vehicle. The control unit 103 can, for example, adapt a seat adjustment of the seat 101 or activate an adjusting device (not shown), for example, arranged in the footrest 104.

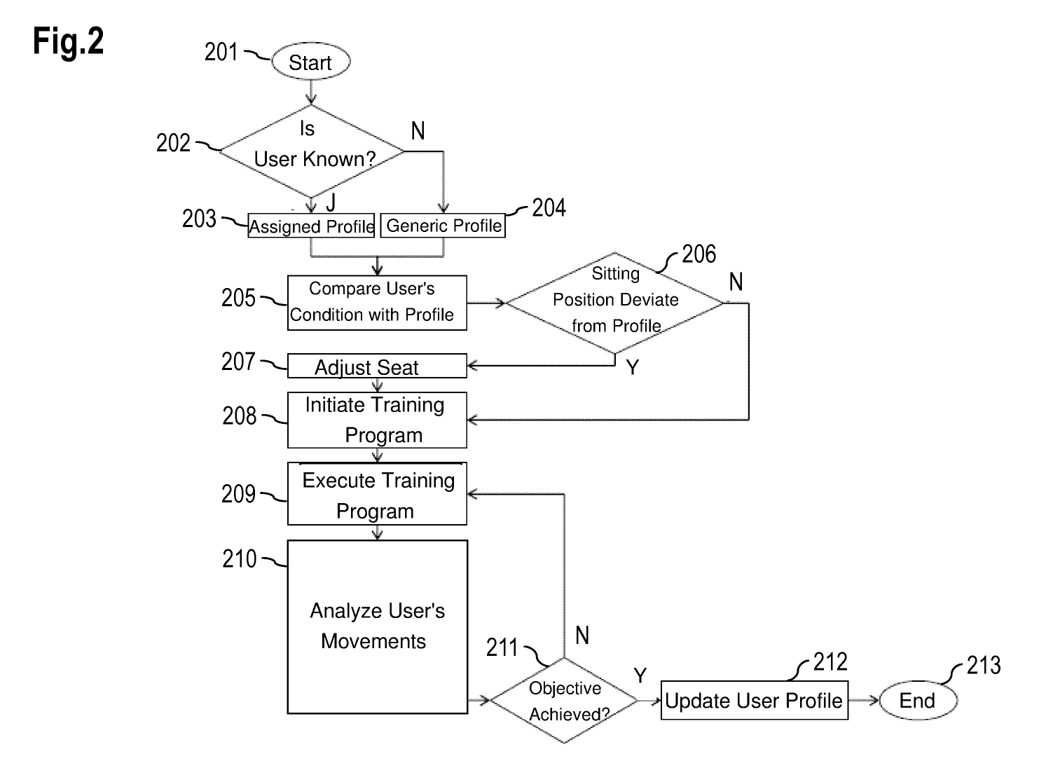

FIG. 2 shows a flow chart for a process used as an example, which uses image data of the camera 102.

After the start 201 of the process, a user is defined in Step 202 in that the image data of the person imaged by way of the camera are analyzed as to whether this person can be assigned to an already known user. If a known user is identified, a profile assigned to this user is downloaded in a Step 203. If no known user can be identified, a general user profile will be used in a Step 204.

A user's condition can be determined in that a sitting position and vital parameters, such as heart beat, body temperature, breathing, etc., are determined by means of image data. In a Step 205, the determined condition can be matched with the downloaded user profile.

In a Step 206, it is determined whether the user's sitting position determined by means of the image data deviates from the sitting position stored in the user profile. If this is yes, in a Step 207, the seat is adjusted by means of at least one adjusting device such that the user takes up a predefined sitting position. Subsequently, a branching to a Step 208 takes place, in which a program, for example, a training program is initialized. The training program may, for example, be preset according to the downloaded user profile. Thus, for example, a training or fitness condition, a previously concluded or a not yet concluded and/or a degree of difficulty or an intensity of the training may be preadjusted

If it is determined in Step 206 that the sitting position corresponds to the sitting position stored in the user profile, a direct branching to Step 208 can take place.

It is also contemplated that the sitting position is a training position. For example, the user can store a training position for the seat adjustment. If a user profile is active, this training position can automatically be downloaded during the activating of the training program. Optionally, an adjustment in a menu can be provided for this purpose. If no user profile is active (if, for example, a general profile is used), the system can determine by means of the data of the sitting position and of the camera whether a carrying-out of the exercise is conceivable in the adjusted position. If this is not so, the user can be prompted to change the seat adjustment or a notification can be issued that the training program does not optimally function in the adjusted seat position.

In a Step 209, the program is started according to its presetting (for example, with correspondingly adjusted parameters). For example, the program may be a training program in which the user carries out body training as a function of instructions which are emitted by way of the display unit 109 and/or the loudspeaker 110.

Subsequent reactions, particularly the user's movements, are captured by the camera 102, the sensor units 105, 106 and pressure sensors 107. Corresponding image data of the camera 102 and additionally corresponding signals of the sensor units 105, 106 and/or pressure sensors 107 can be analyzed in a Step 210 or compared with predefined values. It can basically be determined whether the detected signals and/or values derived therefrom meet predefined requirements. Deviations (for example, from a training objective) can also be determined and be acknowledged to the user.

In Step 211, it can be examined whether a predefined objective (for example, a training goal or a partial goal of a training) was reached. If not, a branching back to Step 209 can take place, in which case, the training can be implemented again or continued with adapted parameters. If the object was reached, in a Step 212, the user profile can be updated by information concerning the achieved objective and can be stored, before the program is terminated in a Step 213.

It is also contemplated that it is examined in Step 211 whether the user would like to end the program. If that is so, Step 212 will follow; otherwise, Step 209 will follow, as required, with adapted parameters.

According to further developments, a portion of the described process steps can be eliminated, for example, the determination of a portion of the parameters when determining the user's condition.

According to a further development, a determination of the user can take place in that the user is registered by way of an interface, which is provided in addition to the camera.

According to a further development, the determining and downloading of a user profile can be eliminated and instead, independently of the user and directly, a program can be activated, for example, for adjusting the seat position and/or the training program.

A desired-actual value adjustment can be implemented with respect to the user's training movement. A desired-actual value adjustment can take place with respect to the pressure-measuring values in the seat, in the footrest or floor mat, in armrests and/or in other body contact surfaces. For example, each sensor system in the vehicle or the control unit or another computer unit can be adjusted by means of the visual image data of the camera 102.

The acquired data can be processed and/or stored in a computer, (for example, in an electronic control unit) of the vehicle. Likewise, the data can be stored on a data storage device or in a network memory, for example, a so-called Cloud. User profiles can also be established, which are accessible, for example, to a community of users. For example, users can be additionally motivated by way of a competition. In this manner, they also can observe the training sequence.

An adjustment of the seat or of an adjustable footrest can take place by way of differently designed adjusting devices; for example, mechanically by use of a spring; electrically, hydraulically or electro-mechanically by use of corresponding controllable drives. Controllable adjusting devices can also be further developed for adjusting different positions of the seat or of an adjustable footrest and/or by use of different forces.

The camera supplies information which can be used for improving sitting comfort, occupant safety or as a component of a system for body training in the vehicle. The sensor units 105, 106 and pressure sensors 107 supply information which can be used for improving sitting comfort, occupant safety or as a component of a system for body training in the vehicle. The camera and the sensor units 105, 106 and pressure sensors 107 therefore permit an improved interaction between the user and the electronic system of the vehicle. Thus, for example, different training exercises can be offered, for example, for the leg muscles or for games.

### LIST OF REFERENCE NUMBERS

101 Seat

102 Camera

103 Control unit

104 Footrest

105 Sensor unit

106 Sensor unit

107 Pressure sensors

108 Memory with database (databank)

109 Display unit

110 Loudspeaker

201 to 213 Process steps

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. An arrangement for use with a vehicle, comprising:
a camera arranged in the vehicle;
a processing device coupled with the camera, wherein
a user's movement is determined in the vehicle via the camera and the processing device; and
an interactive program is implemented for the user as a function of the user's movement.

2. The arrangement according to claim 1, wherein
the camera is arranged in the vehicle such that the user is optically captured, and
image data of the optically captured user from the camera is analyzed via the processing unit.

3. The arrangement according to claim 1, further comprising at least one pressure sensor, wherein signals of the at least one pressure sensor are analyzed via the processing unit.

4. The arrangement according to claim 1, wherein a user's condition is determined via the camera and the processing unit.

5. The arrangement according to claim 3, wherein a user's condition is determined via the camera and the processing unit.

6. The arrangement according to claim 1, wherein the user's movement is compared with a predefined value.

7. The arrangement according to claim 1, further comprising at least one of a display unit and a loudspeaker, wherein the at least one of the display unit and the loudspeaker are activated via the processing unit.

8. The arrangement according to claim 1, further comprising an adjusting device activated as a function of the user's movement.

9. The arrangement according to claim 8, wherein the adjusting device is operatively configured for training of the user.

10. The arrangement according to claim 8, wherein the adjusting device adjusts a seat position of a seat in the vehicle.

11. A motor vehicle, comprising an arrangement according to claim 1.
